(19) United States
(12) Patent Application Publication
(10) Pub. No.: US 2007/0276141 A1
(43) Pub. Date: Nov. 29, 2007

(54) Preparation of Pharmaceutical Salts of [1,4] - Bipiperidine

(76) Inventors: Howard Else, Leicestershire (GB); Richard Evans, Leicestershire (GB); Peter Morgan, Leicestershire (GB); Philip O'keefe, Leicestershire (GB); Matthew Perry, Leicestershire (GB); Phil Plumb, Leicestershire (GB); Mark Purdie, Leicestershire (GB); Brian Springthorpe, Leicestershrie (GB); Gerald Steele, Leicestershire (GB)

Correspondence Address: FISH & RICHARDSON P.C., P.O BOX 1022, MINNEAPOLIS, MN, 55440-1022, US

(21) Appl. No.: 10/578,236

(22) PCT Filed: Nov. 3, 2004
(86) PCT No.: PCT/SE04/01590

§ 371 Date: Feb. 14, 2007

(30) Foreign Application Priority Data
Nov. 7, 2003 (SE) 0302956-8

(51) Int. Cl. C07D 211/40

(52) U.S. Cl. 546/188

(57) ABSTRACT

The invention provides anhydrous and hydrated forms of sodium salt of N-[[4-(3,4-dichlorophenoxy)[1,4′-bipiperidin]-1′-yl]carbonyl]-4-methyl-benzenesulfonamide and crystalline forms of N-[[4-(3,4-dichlorophenoxy)[1,4′-bipiperidin]-1′-yl]carbonyl]-4-methyl-benzenesulfonamide; and such compounds are modulators of chemokine (especially CCR3) activity and are especially useful for treating asthma and/or rhinitis.

The present invention concerns forms of N-[[4-(3,4-dichlorophenoxy)[1,4′-bipiperidin]-1′-yl]carbonyl]-4-methyl-benzenesulfonamide, and solvated (such as hydrated) and anhydrous forms of its sodium salt; to processes for preparing such forms; to pharmaceutical compositions comprising such form; and to the use of such forms as an active therapeutic agent in the treatment of a chemokine (such as CCR3) or H1 mediated disease state.

N-[[4-(3,4-Dichlorophenoxy)[1,4′-bipiperidin]-1′-yl]carbonyl]-4-methyl-benzenesulfonamide: 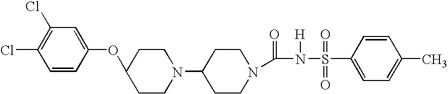
and its sodium salt are disclosed in Example 10A of WO 03/004487. In Example 10A of WO 03/004487 the sodium salt of N-[[4-(3,4-dichlorophenoxy)[1,4′-bipiperidin]-1′-yl]carbonyl]4-methyl-benzenesulfonamide is disclosed as an anhydrous form (hereinafter called Anhydrous Form A). The X-ray powder diffraction pattern of Anhydrous Form A is provided below as FIG. A. N-[[4-(3,4-Dichlorophenoxy)[1,4′-bipiperidin]-1′-yl]carbonyl]-4-methyl-benzenesulfonamide is presented in Annual Reports in Medicinal Chemistry (2003) 38 page 135.

In this document a reference to the sodium salt of N-[[4-(3,4-dichlorophenoxy)[1,4′-bipiperidin]-1′-yl]carbonyl]-4-methyl-benzenesulfonamide is a reference to the mono-sodium salt of N-[[4-(3,4-dichlorophenoxy)[1,4′-bipiperidin]-1′-yl]carbonyl]-4-methyl-benzenesulfonamide.

It has now surprisingly been found that there are two further anhydrous forms (Anhydrous Form B and Anhydrous Form C) of the sodium salt of N-[[4-(3,4-dichlorophenoxy)[1,4′-bipiperidin]-1′-yl]carbonyl]-4-methyl-benzenesulfonamide.

Thus, the present invention provides an anhydrous form of the sodium salt of N-[[4-(3,4-dichlorophenoxy)[1,4′-bipiperidin]-1′-yl]carbonyl]-4-methyl-benzenesulfonamide Anhydrous Form B having an X-ray powder diffraction pattern containing specific peaks at: 3.8 (±0.1°), 7.5 (±0.1°), 11.2 (±0.1°), 13.0 (±0.1°), 13.8 (±0.1°), 15.0 (±0.1°), 15.7 (±0.1°), 18.8 (±0.1°), 20.2 (±0.1°), 21.7 (±0.1°), 22.6 (±0.1°) and 30.2 (±0.1°) 2θ.

The present invention also provides an anhydrous form of the sodium salt of N-[[4-(3,4-dichlorophenoxy)[1,4′-bipiperidin]-1′-yl]carbonyl]-4-methyl-benzenesulfonamide Anhydrous Form C having an X-ray powder diffraction pattern containing specific peaks at: 4.3 (±0.1°), 8.5 (±0.1°), 14.6 (±0.1°), 15.3 (±0.1°), 16.1 (±0.1°), 17.4 (±0.1°), 18.7 (±0.1°), 20.5 (±0.1°), 22.1 (±0.1°), 22.6 (±0.1°), 23.1 (±0.1°) and 29.6 (±0.1°) 2θ.

It has now surprisingly also been found that there are four hydrated forms (Hydrate Form A, Hydrate Form B, Hydrate Form C and Hydrate Form D) of the sodium salt of N-[[4-(3,4-dichlorophenoxy)[1,4′-bipiperidin]-1′-yl]carbonyl]-4-methyl-benzenesulfonamide.

Thus, the present invention provides a hydrated form of the sodium salt of N-[[4-(3,4-dichlorophenoxy)[1,4′-bipiperidin]-1′-yl]carbonyl]-4-methyl-benzenesulfonamide Hydrate Form A having an X-ray powder diffraction pattern containing specific peaks at: 4.2 (±0.1°), 8.2 (±0.1°), 8.5 (±0.1°), 9.1 (±0.1°), 11.5 (±0.1°), 12.7 (±0.1°), 14.8 (±0.1°), 15.4 (±0.1°), 16.6 (±0.1°), 17.4 (±0.1°), 17.7 (±0.1°), 18.2 (±0.1°), 20.4 (±0.1°), 23.2 (±0.1°), 29.1 (±0.1°) and 29.8 (±0.1°) 2θ. In one particular aspect the present invention provides Hydrate Form A of the sodium salt of N-[[4-(3,4-dichlorophenoxy)[1,4′-bipiperidin]-1′-yl]carbonyl]-4-methyl-benzenesulfonamide wherein the water of crystallization is 3-10% w/w.

The present invention also provides a hydrated form of the sodium salt of N-[[4-(3,4-dichlorophenoxy)[1,4′-bipiperidin]-1′-yl]carbonyl]-4-methyl-benzenesulfonamide Hydrate Form B having an X-ray powder diffraction pattern containing specific peaks at: 4.5 (±0.1°), 7.3 (±0.1°), 8.3 (±0.1°), 13.3 (±0.1°), 14.5 (±0.1°), 14.8 (±0.1°), 15.4 (±0.1°), 16.6 (±0.1°), 18.7 (±0.1°), 20.2 (±0.1°), 21.1 (±0.1°), 21.5 (±0.1°), 21.9 (±0.1°), 22.3 (±0.1°), 23.5 (±0.1°) and 24.9 (±0.1°) 2θ. In one particular aspect the present invention provides Hydrate Form B of the sodium salt of N-[[4-(3,4-dichlorophenoxy)[1,4′-bipiperidin]-1′-yl]carbonyl]-4-methyl-benzenesulfonamide wherein the water of crystallization is 5-7% w/w.

The present invention also provides a hydrated form of the sodium salt of N-[[4-(3,4-dichlorophenoxy)[1,4′-bipiperidin]-1′-yl]carbonyl]-4-methyl-benzenesulfonamide Hydrate Form C having an X-ray powder diffraction pattern containing specific peaks at: 4.2 (±0.1°), 7.5 (±0.1°), 8.0 (±0.1°), 11.4 (±0.1°), 12.5 (±0.1°), 15.1 (±0.1°), 15.8 (±0.1°), 17.7 (±0.1°), 18.9 (±0.1°), 20.5 (±0.1°), 21.1 (±0.1°), 22.7 (±0.1°), 24.6 (±0.1°), 26.1 (±0.1°), 27.8 (±0.1°) and 29.2 (±0.1°) 2θ. In one particular aspect the present invention provides Hydrate Form C of the sodium salt of N-[[4-(3,4-dichlorophenoxy)[1,4′-bipiperidin]-1′-yl]carbonyl]-4-methyl-benzenesulfonamide wherein the water of crystallization is 3-10% w/w.

The present invention also provides a hydrated form of the sodium salt of N-[[4-(3,4-dichlorophenoxy)[1,4′-bipiperidin]-1′-yl]carbonyl]-4-methyl-benzenesulfonamide Hydrate Form D having an X-ray powder diffraction pattern containing specific peaks at: 8.8 (±0.1°), 10.5 (±0.1°), 11.8 (±0.1°), 12.9 (±0.1°), 15.6 (±0.1°), 17.1 (±0.1°), 18.9 (±0.1°), 20.8 (±0.1°), 23.3 (±0.1°), 25.6 (±0.1°), 26.1 (±0.1°), 26.9 (±0.1°), 28.1 (±0.1°), 30.6 (±0.1°), 32.5 (±0.1°) and 33.1 (±0.1°) 2θ.

The present invention also provides a solvated form of the sodium salt of N-[[4-(3,4-dichlorophenoxy)[1,4′-bipiperidin]-1′-yl]carbonyl]-4-methyl-benzenesulfonamide (Solvated Form E) having an X-ray powder diffraction pattern containing specific peaks at: 3.6 (±0.1°), 7.1 (±0.1°), 8.3 (±0.1°), 9.3 (±0.1°), 9.8 (±0.1°), 14.1 (±0.1°), 15.9 (±0.1°), 17.7 (±0.1°), 18.6 (±0.1°), 19.3 (±0.1°), 21.7 (±0.1°), 23.1 (±0.1°), 24.1 (±0.1°), 25.0 (±0.1°), 25.8 (±0.1°) and 26.3 (±0.1°) 2θ.

Hydrate Form A is, surprisingly, easier to manufacture than Hydrate Forms B and D and Solvated Form E; and its manufacture is more economic in terms of use of resources, materials and time than the manufacture of Hydrate Forms B and D and Solvated Form E. Hydrate Forms A and C are surprisingly more stable than Hydrate Forms B and D and Solvated Form E.

Further, it has now surprisingly been found that there are two polymorphic forms of N-[[4-(3,4-dichlorophenoxy)[1,4′-bipiperidin]-1′-yl]carbonyl]-4-methyl-benzenesulfonamide (Form A and Form B).

Thus, the present invention provides N-[[4-(3,4-dichlorophenoxy)[1,4′-bipiperidin]-1′-yl]carbonyl]-4-methyl-benzenesulfonamide (Form A) having an X-ray powder diffraction pattern containing specific peaks at: 7.3 (±0.1°), 8.5 (±0.1°), 10.6 (±0.1°), 13.4 (±0.1°), 14.7 (±0.1°), 15.4 (±0.1°), 15.9 (±0.1°), 19.9 (±0.1°), 20.2 (±0.1°), 21.7 (±0.1°), 25.8 (±0.1°) and 26.6 (±0.1°) 2θ.

The present invention also provides N-[[4-(3,4-dichlorophenoxy)[1,4′-bipiperidin]-1′-yl]carbonyl]-4-methyl-benzenesulfonamide (Form B) having an X-ray powder diffraction pattern containing specific peaks at: 9.9 (±0.1°), 10.5 (±0.1°), 11.0 (±0.1°), 11.6 (±0.1°), 13.3 (±0.1°), 13.9 (±0.1°), 14.9 (±0.1°), 18.0 (±0.1°), 19.0 (±0.1°), 20.4 (±0.1°), 22.2 (±0.1°) and 23.0 (±0.1°) 2θ.

The Anhydrous Form B of the sodium salt of N-[[4-(3,4-dichlorophenoxy)[1,4′-bipiperidin]-1′-yl]carbonyl]-4-methyl-benzenesulfonamide can be prepared as follows. 4-(3,4-Dichlorophenoxy)-1,4′-bipiperidine is reacted with 4-methylbenzenesulfonyl isocyanate in a suitable solvent (for example dichloromethane) keeping the temperature below 30° C. (for example at a temperature in the range 10-30° C.) Solid N-[[4(3,4-dichlorophenoxy)[1,4′-bipiperidin]-1′-yl]carbonyl]-4-methyl-benzenesulfonamide forms and is separated and then dissolved in aqueous sodium hydroxide. The aqueous solution is extracted with a suitable organic solvent (for example dichloromethane), the organic extracts are combined, the volume of solvent reduced and sodium salt of N-[[4-(3,4-dichlorophenoxy)[1,4′-bipiperidin]-1′-yl]carbonyl]-4-methyl-benzenesulfonamide crystallizes from solution. The salt may be recrystallized from ethanol-water. The salt is suspended in aqueous sodium hydroxide and dichloromethane, the organic layer is separated and filtered to leave a residue which is triturated with water and then dried in the presence of phosphorus pentoxide under reduced pressure (such as below 50 mm Hg), for example at a temperature in the range 20-60° C.

Alternatively, the Anhydrous Form B of the sodium salt of N-[[4-(3,4-dichlorophenoxy)[1,4′-bipiperidin]-1′-yl]carbonyl]-4-methyl-benzenesulfonamide can be prepared by taking the Hydrate Form A of the sodium salt of N-[[4-(3,4-dichlorophenoxy)[1,4′-bipiperidin]-1′-yl]carbonyl]-4-methyl-benzenesulfonamide and drying it in the presence of phosphorus pentoxide under reduced pressure (such as below 50 mm Hg), for example at a temperature in the range 20-60° C.

Alternatively, the Anhydrous Form B of the sodium salt of N-[[4-(3,4-dichlorophenoxy)[1,4′-bipiperidin]-1′-yl]carbonyl]-4-methyl-benzenesulfonamide can be prepared by taking the Hydrate Form A of the sodium salt of N-[[4-(3,4-dichlorophenoxy)[1,4′-bipiperidin]-1′-yl]carbonyl]-4-methyl-benzenesulfonamide and heating it from ambient temperature (that is room temperature, such as 10-30° C.) to 100° C., for example under an atmosphere of nitrogen.

The Anhydrous Form C of the sodium salt of N-[[4-(3,4-dichlorophenoxy)-[1,4′-bipiperidin]-1′-yl]carbonyl]-4-methyl-benzenesulfonamide can be prepared by taking the Hydrate Form B of the sodium salt of N-[[4-(3,4-dichlorophenoxy)[1,4′-bipiperidin]-1′-yl]carbonyl]-4-methyl-benzenesulfonamide and heating it from ambient temperature (that is room temperature, such as 10-30° C.) to 100° C., for example under an atmosphere of nitrogen.

The Hydrate Form A of the sodium salt of N-[[4-(3,4-dichlorophenoxy)[1,4′-bipiperidin]-1′-yl]carbonyl]-4-methyl-benzenesulfonamide can be prepared as follows. 4-(3,4-Dichlorophenoxy)-1,4′-bipiperidine is reacted with 4-methylbenzenesulfonyl isocyanate in a suitable solvent (for example tetrahydrofuran) at ambient temperature (such as a temperature in the range 10-30° C.) to form N-[[4-(3,4-dichlorophenoxy)[1,4′-bipiperidin]-1′-yl]carbonyl]-4-methyl-benzenesulfonamide in the suitable solvent; and:
a. concentrated aqueous sodium hydroxide solution (for example 8-12N) is added followed by water. The resulting mixture may then be stirred to allow the sodium salt of N-[[4-(3,4-dichlorophenoxy)[1,4′-bipiperidin]-1′-yl]carbonyl]-4-methyl-benzenesulfonamide, possibly contaminated with suitable solvent, to precipitate out, the said crude product is recrystallized from water and Hydrate Form A remains after filtration and drying, or, alternatively, the suitable solvent can be distilled off and the sodium salt of N-[[4-(3,4-dichlorophenoxy)[1,4′-bipiperidin]-1′-yl]carbonyl]-4-methyl-benzenesulfonamide allowed to precipitate from the aqueous and desired Hydrate Form A remains after filtration and drying; OR,
b. water is added and N-[[4-(3,4-dichlorophenoxy)[1,4′-bipiperidin]-1′-yl]carbonyl]-4-methyl-benzenesulfonamide precipitates. The N-[[4-(3,4-dichlorophenoxy)[1,4′-bipiperidin]-1′-yl]carbonyl]-4-methyl-benzenesulfonamide is mixed with water, heated to a temperature in the range 30-60° C., concentrated aqueous sodium hydroxide solution (for example 8-12N) is added and the mixture cooled with the sodium salt of N-[[4-(3,4-dichlorophenoxy)[1,4′-bipiperidin]-1′-yl]carbonyl]-4-methyl-benzenesulfonamide precipitating and Hydrate Form A remains after filtration and drying.

Alternatively, the Hydrate Form A of the sodium salt of N-[[4-(3,4-dichlorophenoxy)[1,4′-bipiperidin]-1′-yl]carbonyl]-4-methyl-benzenesulfonamide can be prepared as follows. 4-(3,4-Dichlorophenoxy)-1,4′-bipiperidine is reacted with 4-methylbenzenesulfonyl isocyanate in a suitable organic solvent (for example chlorobenzene or a mixture of tetrahydrofuran-and toluene) at a temperature in the range 10-50° C. to form N-[[4-(3,4-dichlorophenoxy)[1,4′-bipiperidin]-1′-yl]carbonyl]-4-methyl-benzenesulfonamide in the suitable solvent; and concentrated aqueous sodium hydroxide solution (for example 8-12N) is added. The resulting mixture is heated (for example to a temperature in the range 50-80° C.) and the aqueous phase separated. IMS (Industrial Methylated Spirit) (for example IMS 74 OP) and, optionally, toluene are added to the aqueous phase and the resulting mixture is cooled to 0-10° C. Solid forms, is filtered off and dried (for example at 15-40° C., 15-40 mbar) to provide Hydrate Form A.

Alternatively, the Hydrate Form A of the sodium salt of N-[[4-(3,4-dichlorophenoxy)[1,4′-bipiperidin]-1′-yl]carbonyl]-4-methyl-benzenesulfonamide can be prepared by mixing N-[[4-(3,4-dichlorophenoxy)[1,4′-bipiperidin]-1′-yl]carbonyl]-4-methyl-benzenesulfonamide Form B and aqueous sodium hydroxide, heating the mixture (such as to 40-60° C.) and then extracting the cooled mixture with dichloromethane. The volume of solvent of combined organic extracts may be reduced and the extracts are cooled (such as to −10 to 10° C.) for example with stirring, and the sodium salt of N-[[4-(3,4-dichlorophenoxy)[1,4′-bipiperidin]-1′-yl]carbonyl]-4-methyl-benzenesulfonamide precipitates, and Hydrate Form A remains after filtration and drying. Hydrate Form A may be dried under reduced pressure (for example below 50 mm Hg) at 30-50° C.

Alternatively, Hydrate Form A can be prepared by drying a sample of Hydrate Form D under reduced pressure (for example below 50 mm Hg) at a temperature in the range 10-100° C. (for example 20-50° C.).

Alternatively, Hydrate Form A can be prepared by drying a sample of Solvated Form E at atmospheric pressure at a temperature in the range 0-30° C.

The Hydrate Form B of the sodium salt of N-[[4-(3,4-dichlorophenoxy)[1,4′-bipiperidin]-1′-yl]carbonyl]-4-methyl-benzenesulfonamide can be prepared by mixing a solution of 4-(3,4-dichlorophenoxy)-1,4′-bipiperidine in tetrahydrofuran with a solution of 4-methylbenzenesulfonyl isocyanate in tetrahydrofuran at a temperature in the range 15-35° C. Aqueous sodium hydroxide solution (such as concentrated (for example 10N); 1 equivalent), is added and the Hydrate Form B precipitates from the reaction mixture.

The Hydrate Form C of the sodium salt of N-[[4-(3,4-dichlorophenoxy)[1,4′-bipiperidin]-1′-yl]carbonyl]-4-methyl-benzenesulfonamide can be prepared by dissolving the sodium salt of N-[[4-(3,4-dichlorophenoxy)[1,4′-bipiperidin]-1′-yl]carbonyl]-4-methyl-benzenesulfonamide in a mixture of water and acetone (for example in the v/v ratio of about 1:4) at reflux and allowing the solution to cool to room temperature and then cooling it to around 0° C. The Hydrate Form C crystallizes from solution during the cooling.

Alternatively, Hydrate Form C can be prepared by drying a sample of Solvated Form E reduced pressure (for example below 200 mbar) at a temperature in the range 10-100° C. (for example 20-50° C.).

Hydrate Form D can be prepared by heating a mixture of the sodium salt of N-[[4-(3,4-dichlorophenoxy)[1,4′-bipiperidin]-1′-yl]carbonyl]-4-methyl-benzenesulfonamide in a mixture of water and 2-propanol (for example in the v/v ratio of about 1:1) to 50-80° C. to form a solution. The solution is cooled (for example at a rate of 0.3-0.7° C./min; such as about 0.5° C./min) to 0-10° C., stirred and then filtered. The residue is washed with water and contains Hydrate Form D.

Solvated Form E can be prepared by heating a mixture of the sodium salt of N-[[4-(3,4-dichlorophenoxy)[1,4′-bipiperidin]-1′-yl]carbonyl]-4-methyl-benzenesulfonamide in a mixture of water, IMS (for example IMS 74OP) and toluene (for example in the v/v ratio of about 40:20:3) to 50-80° C. to form a solution. The solution is cooled (for example at a rate of 0.3-0.7° C./min; such as about 0.5° C./min) to 0-10° C., stirred and then filtered to leave a residue that contains Solvated Form E.

N-[[4-(3,4-Dichlorophenoxy)[1,4′-bipiperidin]-1′-yl]carbonyl]-4-methyl-benzenesulfonamide Form A can be prepared by crystallizing N-[[4-(3,4-dichlorophenoxy)-[1,4′-bipiperidin]-1′-yl]carbonyl]-4-methyl-benzenesulfonamide from ethanol and then purifying the crystallized product using reverse phase chromatography eluting with a mixture of aqueous ammonia and acetonitrile. The desired fractions are combined, freeze dried and the residue triturated with acetonitrile and then dried under reduced pressure at ambient temperature (10 to 30° C.).

Alternatively, N-[[4-(3,4-dichlorophenoxy)[1,4′-bipiperidin]-1′-yl]carbonyl]-4-methyl-benzenesulfonamide Form A can be prepared by mixing N-[[4-(3,4-dichlorophenoxy)[1,4′-bipiperidin]-1′-yl]carbonyl]-4-methyl-benzenesulfonamide Form B and acetonitrile and heating the mixture to 40-60° C. The solid from the slurry so formed is dried under reduced pressure, for example at a temperature in the range 30 to 50° C.

N-[[4-(3,4-Dichlorophenoxy)[1,4′-bipiperidin]-1′-yl]carbonyl]-4-methyl-benzenesulfonamide Form B can be prepared by mixing 4-methylbenzenesulfonyl isocyanate and 4-(3,4-dichlorophenoxy)-1,4′-bipiperidine in dichloromethane. The mixture is stirred. Then:
1. Water is added. The organic layer is separated and allowed to stand and product crystallizes from solution. The solid is collected and can be dried under reduced pressure (such as below 50 mm Hg) for example at a temperature in the range 30-50° C.
   OR,
2. Solid precipitates from solution. The solid may be washed with dichloromethane. The solid is dried under reduced pressure (such as below 50 mm Hg) for example at a temperature in the range 30-50° C.

In further aspects the present invention provides processes for the preparation of the compounds of the invention.

The compounds of the invention have activity as pharmaceuticals, in particular as modulators of chemokine receptor (especially CCR3) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

Examples of these conditions are:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis; sarcoidosis; farmer's lung and related diseases; nasal polyposis; fibroid lung, idiopathic interstitial pneumonia, antitussive activity, treatment of chronic cough associated with inflammatory conditions of the airways or iatrogenic induced cough;
(2) (bone and joints) arthrides including rheumatic, infectious, autoimmune, seronegative spondyloarthropathies (such as ankylosing spondylitis, psoriatic arthritis or Reiter's disease), Behget's disease, Sjogren's syndrome or systemic sclerosis;
(3) (skin and eyes) psoriasis, atopic dermatitis, contact dermatitis or other eczematous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allograft rejection) acute and chronic following, for example, transplantation of kidney, heart, liver, lung, bone marrow, skin or cornea; or chronic graft versus host disease; and/or
(6) (other tissues or diseases) Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura or disorders of the menstrual cycle.

The compounds of the invention are also H1 antagonists and may be used in the treatment of allergic disorders.

The compounds of the invention may also be used to control a sign and/or symptom of what is commonly referred to as a cold (for example a sign and/or symptom of a common cold or influenza or other associated respiratory virus infection).

According to a further feature of the invention there is provided a compound of the invention for use in a method of treatment of a warm blooded animal (such as man) by therapy (including prophylaxis).

According to a further feature of the present invention there is provided a method for modulating chemokine receptor activity (especially CCR3 receptor activity), or antagonising H1, in a warm blooded animal, such as man, in need of such treatment, which comprises administering to said animal an effective amount of a compound of the invention.

The invention also provides a compound of the invention for use as a medicament.

In another aspect the invention provides the use of a compound of the invention in the manufacture of a medicament for use in therapy (for example modulating chemokine receptor activity (especially CCR3 receptor activity), or antagonising H1, in a warm blooded animal, such as man).

The invention further provides the use of a compound of the invention in the manufacture of a medicament for use in the treatment of:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis; sarcoidosis; farmer's lung and related diseases; nasal polyposis; fibroid lung, idiopathic interstitial pneumonia, antitussive activity, treatment of chronic cough associated with inflammatory conditions of the airways or iatrogenic induced cough;
(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;
(3) (skin and eyes) psoriasis, atopic dermatitis, contact dermatitis or other eczematous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allograft rejection) acute and chronic following, for example, transplantation of kidney, heart, liver, lung, bone marrow, skin or cornea; or chronic graft versus host disease; and/or
(6) (other tissues or diseases) Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, sezary syndrome, idiopathic thrombocytopenia pupura or disorders of the menstrual cycle;
(7) H1 antagonists and may be used in the treatment of allergic disorders; or,
(8) to control a sign and/or symptom of what is commonly referred to as a cold (for example a sign and/or symptom of a common cold or influenza or other associated respiratory virus infection);
   in a warm blooded animal, such as man.

In a further aspect a compound of the invention is useful in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

In a still further aspect a compound of the invention is useful in the treatment of asthma.

The present invention also provides the use of a compound of the invention in the manufacture of a medicament for use in the treatment of asthma or rhinitis.

The present invention further provides a method of treating a chemokine mediated disease state (especially a CCR3 mediated disease state, especially asthma) in a warm blooded animal, such as man, which comprises administering to a mammal in need of such treatment an effective amount of a compound of the invention.

In order to use a compound of the invention for the therapeutic treatment of a warm blooded animal, such as man, in particular modulating chemokine receptor (for example CCR3 receptor) activity or antagonising H1, said compound is normally formulated in accordance with standard pharmaceutical practice as a pharmaceutical composition.

Therefore in another aspect the present invention provides a pharmaceutical composition which comprises a compound of the invention and a pharmaceutically acceptable adjuvant, diluent or carrier. In a further aspect the present invention provides a process for the preparation of said composition which comprises mixing a compound of the invention with a pharmaceutically acceptable adjuvant, diluent or carrier. Depending on the mode of administration, the pharmaceutical composition may comprise from 0.05 to 99% w (per cent by weight), such as from 0.05 to 80 % w, for example from 0.10 to 70% w, or from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1 g of a compound of the invention.

In another aspect a pharmaceutical composition of the invention is one suitable for intravenous, subcutaneous or intramuscular injection.

Each patient may receive, for example, an intravenous, subcutaneous or intramuscular dose of 0.01 mgkg⁻¹ to 100 mgkg⁻¹ of the compound, preferably in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrates a representative pharmaceutical dosage form containing a compound of the invention (Compound X), for therapeutic or prophylactic use in humans:

| Capsule | mg/capsule |
|---|---|
| Compound X | 10 |
| Lactose Ph.Eur. | 389 |
| Croscarmellose sodium | 100 |
| Magnesium stearate | 1.0 |

Buffers, pharmaceutically-acceptable cosolvents such as polyethylene glycol, polypropylene glycol, glycerol or ethanol or complexing agents such as hydroxy-propyl β-cyclodextrin may be used to aid formulation. The compositions of the invention can be obtained by conventional procedures well known in the pharmaceutical art. Tablets may be enteric coated by conventional means, for example to provide a coating of cellulose acetate phthalate.

The invention will now be illustrated by the following non-limiting Examples in which, unless stated otherwise:
(i) when given, ¹H NMR data is quoted and is in the form of delta values for major diagnostic protons, given in parts per million (ppm) relative to tetramethylsilane (TMS) as an internal standard, determined at 300 MHz or 400 MHz using perdeuterio DMSO-D6 (CD₃SOCD₃), methanol-D4 (CD₃OD) or CDCl₃ as the solvent unless otherwise stated;
(ii) mass spectra (MS) were run with an electron energy of 70 electron volts in the chemical ionization (CI) mode using a direct exposure probe; where indicated ionization was effected by electron impact (EI) or fast atom bombardment (FAB) or electrospray (ESI); where values for m/z are given, generally only ions which indicate the parent mass are reported, and unless otherwise stated the mass ion quoted is the positive mass ion—(M+H)⁺;
(iii) the title compounds of the Examples were named using the ACD/Index name program version 4.55 from Advanced Chemistry Development, Inc;
(iv) unless stated otherwise, reverse phase BPLC was conducted using a Symmetry, NovaPak or Xterra reverse phase silica column; and,

(v) the following abbreviations are used:

| | | | |
|---|---|---|---|
| RPHPLC | reverse phase HPLC | THF | tetrahydrofuran |
| XRPD | X-ray powder diffractometry | MeCN | acetonitrile |
| eq. | equivalents | DCM | dichloromethane |
| DMSO | dimethylsulfoxide | m.pt. | melting point |
| IMS | Industrial Methylated Spirits | aq | aqueous |

### METHODS

Method for X-Ray Powder Diffractometry (XRPD)

Analyses were performed on a Siemens model D5000 fitted with a position sensitive detector (PSD), a Philips X'pert Pro fitted with an X'celerator detector or a Rigaku MiniFlex X-ray powder diffractometer fitted with a scintillation detector. Samples (approximately 10 mg) were dispensed as a thin powder layer on a silicon wafer zero-background holder and irradiated with copper K_{α} radiation (λ=1.54056 Å). Reflections were collected between 2 and 40° 2θ, typically at a step size of 0.007° 2θ and a step time of 1 or 2 seconds.

Method for Thermogravimetric Analysis (TGA)

The sample (approximately 5 mg) was dispensed onto the sample pan of a TA Instruments Model Q500 thermogravimetric analyser (TGA). The sample was heated from ambient temperature to 300° C. under an atmosphere of nitrogen at a scan rate of 10° C. min⁻¹.

The following Figures are presented:

FIG. A: XPRD of Anhydrous-Form A

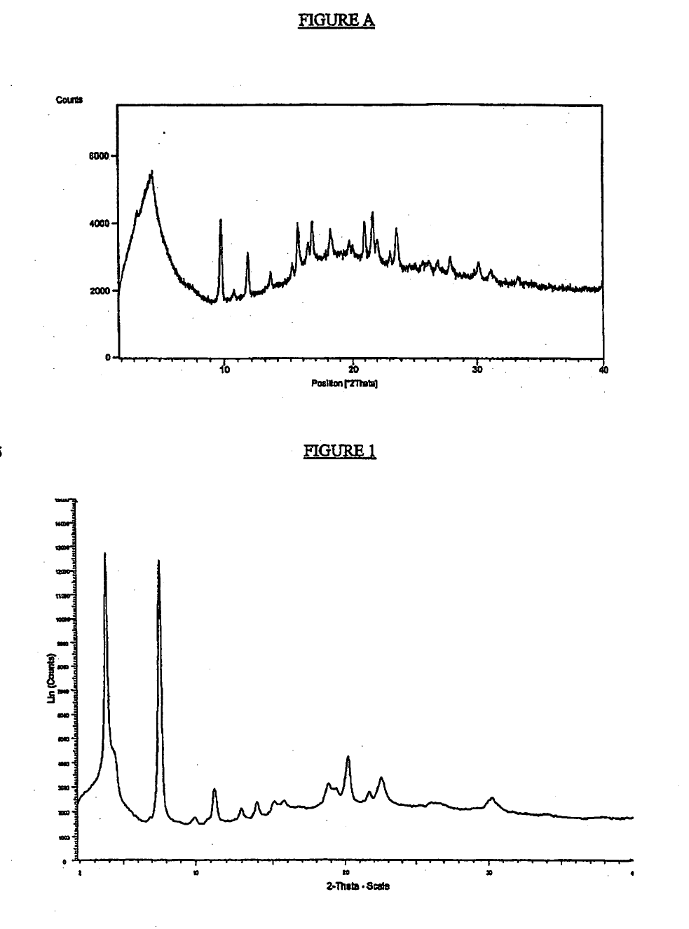

FIG. 1: XRPD of Anhydrous Form B

FIG. 2: XRPD of Anhydrous Form C

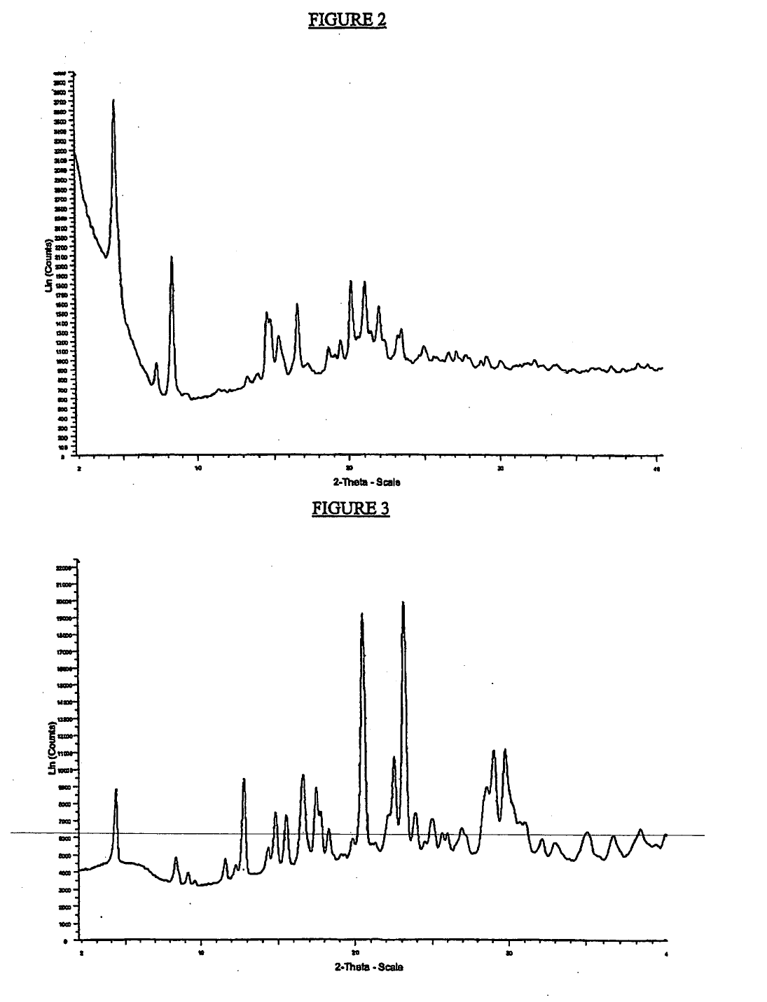

FIG. 3: XRPD of Hydrate Form A

FIG. 4: XRPD of Hydrate From B

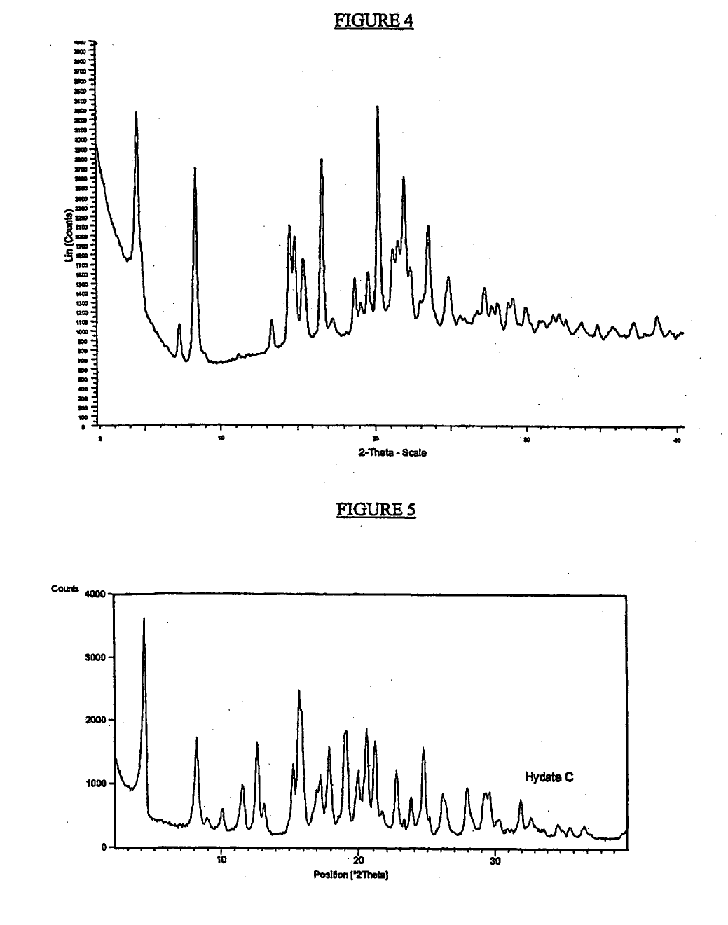

FIG. 5: XRPD of Hydrate From C

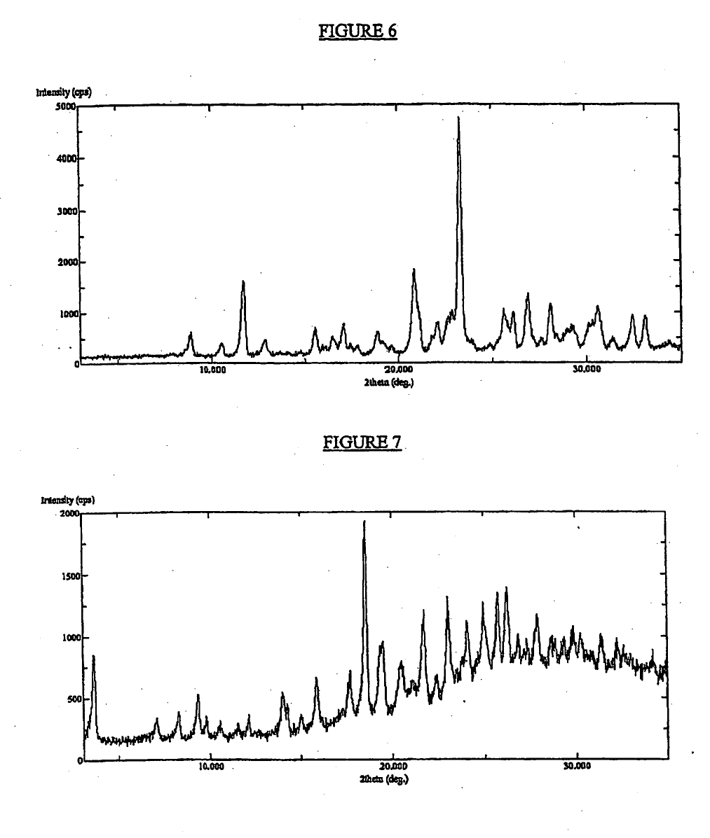

FIG. 6: XRPD of Hydrate From D

FIG. 7: XRPD of Solvated Form E

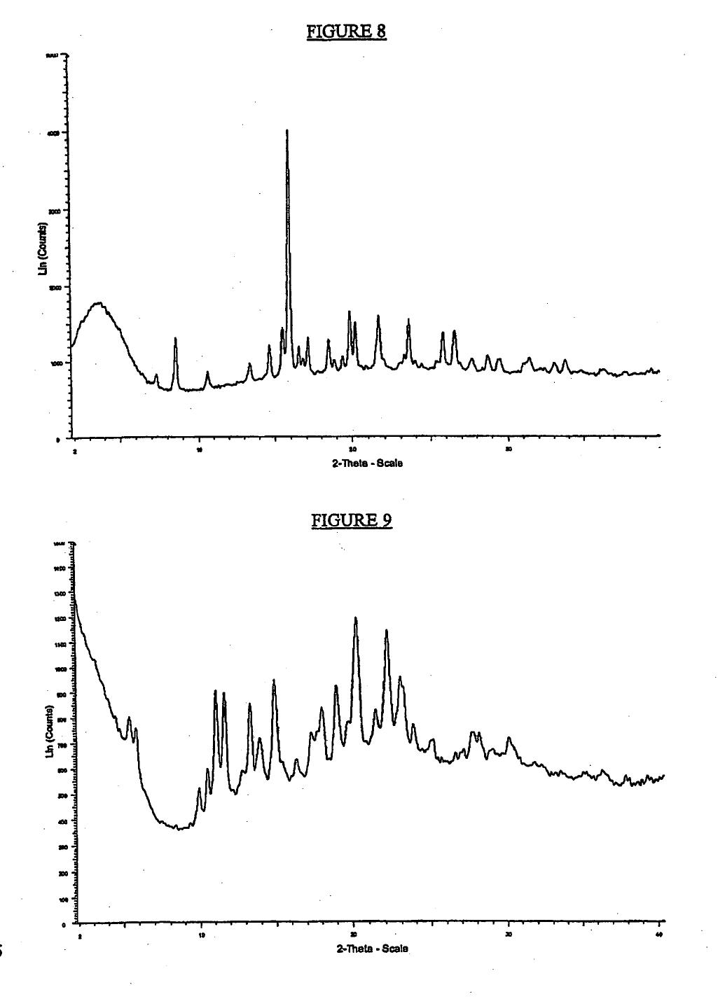

FIG. 8: XRPD of N-[[4-(3,4-dichlorophenoxy)[1,4′-bipiperidin]-1′-yl]carbonyl]-4-methyl-benzenesulfonamide Form A

FIG. 9: XRPD of N-[[4-(3,4-dichlorophenoxy)[1,4′-bipiperidin]-1′-yl]carbonyl]-4-methyl-benzenesulfonamide Form B

XRPD of a sample of Anhydrous Form A is presented in FIG. A. XRPD main reflection peaks are:

| Reflection angle (°2θ) | D-spacing (Å) | Relative intensity (%) |
|---|---|---|
| 9.8 | 9.0 | 64 |
| 10.8 | 8.2 | 4 |
| 11.7 | 7.5 | 35 |
| 13.5 | 6.6 | 13 |
| 15.2 | 5.8 | 16 |
| 15.7 | 5.7 | 47 |
| 16.5 | 5.4 | 29 |
| 16.8 | 5.3 | 47 |
| 18.3 | 4.9 | 37 |
| 19.8 | 4.5 | 27 |
| 20.1 | 4.4 | 21 |
| 21.0 | 4.2 | 38 |
| 21.7 | 4.1 | 47 |
| 22.1 | 4.0 | 26 |
| 23.1 | 3.9 | 15 |
| 23.7 | 3.8 | 37 |
| 24.8 | 3.6 | 6 |
| 25.2 | 3.5 | 6 |
| 25.9 | 3.4 | 10 |
| 26.4 | 3.4 | 11 |
| 27.0 | 3.3 | 11 |
| 28.1 | 3.2 | 16 |
| 29.9 | 3.0 | 7 |
| 30.2 | 3.0 | 14 |
| 31.1 | 2.9 | 9 |
| 33.2 | 2.7 | 7 |
| 39.9 | 2.3 | 8 |

### EXAMPLE 1

This Example illustrates the preparation of the sodium salt of N-[[4-(3,4-dichlorophenoxy)[1,4′-bipiperidin]-1′-yl]carbonyl]-4-methyl-benzenesulfonamide in Anhydrous Form B.

4-(3,4-Dichlorophenoxy)-1,4′-bipiperidine (20 g) was dissolved in dichloromethane (150 ml). 4-Methylbenzenesulfonyl isocyanate (9.3 ml) was added dropwise with cooling to maintain temperature <30° C. After 2 hours a solid was collected and washed with dichloromethane. The solid was dissolved in 0.25M aqueous sodium hydroxide (400 ml); this solution was extracted with dichloromethane thrice. The organic phases were combined and solvent partially evaporated to initiate crystallization, then the product was allowed to crystallize. The solid was collected and recrystallized from ethanol/water (320 ml, 30 ml) and then dried in vacuo.

The resultant solid was suspended in 2M aqueous sodium hydroxide/dichloromethane (100 ml of each); the dichloromethane layer was separated and filtered. The solid so collected was triturated with water, collected and then washed with dichloromethane. Drying in vacuo over P₂O₅ at 40° C. gave the title compound.

m.pt. 229.5-231° C.

Karl Fischer analysis showed 0.26% water. Contains 0.57% w/w moisture by TGA.

XRPD of a sample of Anhydrous Form B is presented in FIG. 1. XRPD main reflection peaks are:

| Reflection angle (°2θ) | D-spacing (Å) | Relative intensity (%) |
|---|---|---|
| 3.8 | 23.5 | 98 |
| 4.4 | 19.9 | 19 |
| 6.7 | 13.1 | 1 |
| 7.5 | 11.8 | 100 |
| 10.0 | 8.9 | 2 |
| 11.2 | 7.9 | 13 |
| 13.0 | 6.8 | 5 |
| 13.8 | 6.4 | 6 |
| 15.0 | 5.9 | 6 |
| 15.7 | 5.6 | 6 |
| 18.8 | 4.7 | 11 |
| 19.4 | 4.6 | 9 |
| 20.2 | 4.4 | 20 |
| 21.7 | 4.1 | 7 |
| 22.6 | 3.9 | 12 |
| 26.1 | 3.4 | 3 |
| 26.9 | 3.3 | 3 |
| 30.2 | 3.0 | 6 |

### EXAMPLE 2

This Example illustrates the preparation of the sodium salt of N-[[4-(3,4-dichlorophenoxy)[1,4′-bipiperidin]-1′-yl]carbonyl]-4-methyl-benzenesulfonamide in Anhydrous Form B.

Sodium salt of N-[[4-(3,4-dichlorophenoxy)[1,4′-bipiperidin]-1′-yl]carbonyl]-4-methyl-benzenesulfonamide Hydrate Form A (see Example 5; 2.63 g) was dried in vacuo at 40° C. in the presence of phosphorus pentoxide for 4 days to give the title compound (2.30 g).

MS [M+H]⁺ (EI) 526/528

¹H NMR δ (CD₃OD) 1.28-1.42 (2H, m), 1.70-1.82 (4H, m), 1.96-2.04 (2H, m), 2.35 (3H, s), 2.43-2.54 (3H, m), 2.56-2.66 (2H, m), 2.80-2.87 (2H, m), 4.34-4.42 (3H, m), 6.87-6.90 (1H, m), 7.09-7.10 (1H, m), 7.19-7.23 (2H, m), 7.35-7.38 (1H, m), 7.75-7.79 (2H, m).

¹³C NMR δ (CD₃OD) 21.4, 29.3, 31.6, 43.2, 47.2, 63.8, 74.5, 117.3, 119.1, 124.7, 128.0, 129.7, 132.1, 133.8, 142.0, 144.0, 158.3, 162.8.

### EXAMPLE 3

This Example illustrates the preparation of the sodium salt of N-[[4-(3,4-dichlorophenoxy)[1,4′-bipiperidin]-1′-yl]carbonyl]-4-methyl-benzenesulfonamide in Anhydrous Form B.

Sodium salt of N-[[4-(3,4-dichlorophenoxy)[1,4′-bipiperidin]-1′-yl]carbonyl]-4-methyl-benzenesulfonamide Hydrate Form A (see Example 5; approximately 10 mg) was dispensed onto the sample pan of a TA Instruments Model Q500 thermogravimetric analyser (TGA). The sample was heated from ambient temperature to 100° C. under an atmosphere of nitrogen at a scan rate of 10° C. min⁻¹. The dried material produced was allowed to cool under ambient laboratory conditions prior to XRPD analysis. Contains 0.22% w/w moisture by TGA

### EXAMPLE 4

This Example illustrates the preparation of the sodium salt of N-[[4-(3,4-dichlorophenoxy)[1,4′-bipiperidin]-1′-yl]carbonyl]-4-methyl-benzenesulfonamide in Anhydrous Form C.

Sodium salt of N-[[4-(3,4-dichlorophenoxy)[1,4′-bipiperidin]-1′-yl]carbonyl]-4-methyl-benzenesulfonamide Hydrate Form B (see Example 10; approximately 10 mg) was dispensed onto the sample pan of a TA Instruments Model Q500 thermogravimetric analyser (TGA). The sample was heated from ambient temperature to 100° C. under an atmosphere of nitrogen at a scan rate of 10° C. min⁻¹. The dried material produced was allowed to cool under ambient laboratory conditions prior to XRPD analysis. No mass loss detected by TGA

XRPD of a sample of Anhydrous Form C is presented in FIG. 2. XRPD main reflection peaks are:

| Reflection angle (°2θ) | D-spacing (Å) | Relative intensity (%) |
|---|---|---|
| 4.2 | 20.8 | 56 |
| 4.9 | 18.0 | 11 |
| 8.5 | 10.4 | 77 |
| 8.9 | 10.0 | 20 |
| 11.4 | 7.8 | 4 |
| 13.0 | 6.8 | 2 |
| 14.6 | 6.1 | 8 |
| 15.3 | 5.8 | 4 |
| 16.1 | 5.5 | 14 |
| 17.1 | 5.2 | 14 |
| 17.4 | 5.1 | 20 |
| 17.6 | 5.0 | 17 |
| 17.7 | 5.0 | 17 |
| 18.7 | 4.7 | 6 |
| 19.7 | 4.5 | 13 |
| 20.5 | 4.3 | 100 |
| 22.1 | 4.0 | 7 |
| 22.6 | 3.9 | 11 |
| 23.1 | 3.8 | 22 |
| 24.2 | 3.7 | 5 |
| 24.5 | 3.6 | 6 |
| 25.0 | 3.6 | 4 |
| 26.2 | 3.4 | 7 |
| 27.3 | 3.3 | 5 |
| 29.6 | 3.0 | 12 |

### EXAMPLE 5

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form A.

To N-[[4-(3,4-dichlorophenoxy)[1,4′-bipiperidin]-1′-yl]carbonyl]-4-methyl-benzenesulfonamide Form B (see Example 13; 8.8 g,) was added 2M aqueous sodium hydroxide (25 ml) and water (50 ml). The suspension was heated to 50° C. to give a solution. The solution was cooled and extracted with DCM (3×50 ml). Solvent (70 ml) was stripped from the combined organic fraction and the remaining solution stirred at 5° C. which caused precipitation. Further DCM (50 ml) was added to aid stirring. The slurry was filtered, washed with DCM (20 ml) and dried in vacuo at 40° C. to give the title compound.

m.pt. 240° C.

MS [M+H]⁺ (EI) 526/528

¹H NMR δ (CD₃OD) 1.28-1.42 (2H, m), 1.70-1.82 (4H, m), 1.96-2.04 (2H, m), 2.35 (3H, s), 2.43-2.54 (3H, m), 2.56-2.66 (2H, m), 2.80-2.87 (2H, m), 4.34-4.42 (3H, m), 6.87-6.90 (1H, m), 7.09-7.10 (1H, m), 7.19-7.23 (2H, m), 7.35-7.38 (1H, m), 7.75-7.79 (2H, m). There is a large water peak at 4.87.

¹³C NMR δ (CD₃OD) 21.4, 29.3, 31.6, 43.2, 47.2, 63.8, 74.5, 117.3, 119.1, 124.7, 128.0, 129.7, 132.1, 133.8, 142.0, 144.0, 158.3, 162.8

Contains 8.80% w/w moisture by TGA; 9.3% w/w water content by Karl Fischer analysis.

XRPD of a sample of Hydrate Form A is presented in FIG. 3. XRPD main reflection peaks are:

| Reflection angle (°2θ) | D-spacing (Å) | Relative intensity (%) |
|---|---|---|
| 4.2 | 21.2 | 100 |
| 8.2 | 10.7 | 14 |
| 8.5 | 10.5 | 11 |
| 9.1 | 9.8 | 10 |
| 9.5 | 9.3 | 3 |
| 11.5 | 7.7 | 13 |
| 12.1 | 7.3 | 8 |
| 12.7 | 7.0 | 91 |
| 14.2 | 6.2 | 7 |
| 14.8 | 6.0 | 19 |
| 15.4 | 5.7 | 15 |
| 16.6 | 5.3 | 26 |
| 17.4 | 5.1 | 19 |
| 17.7 | 5.0 | 15 |
| 18.2 | 4.9 | 16 |
| 19.8 | 4.5 | 6 |
| 20.4 | 4.4 | 64 |
| 21.2 | 4.2 | 12 |
| 22.1 | 4.0 | 10 |
| 22.5 | 3.9 | 23 |
| 23.2 | 3.8 | 98 |
| 24.0 | 3.7 | 7 |
| 24.4 | 3.6 | 6 |
| 24.9 | 3.6 | 9 |
| 25.1 | 3.5 | 9 |
| 25.7 | 3.5 | 8 |
| 26.0 | 3.4 | 7 |
| 26.9 | 3.3 | 7 |
| 27.3 | 3.3 | 6 |
| 28.6 | 3.1 | 13 |
| 29.1 | 3.1 | 21 |
| 29.8 | 3.0 | 18 |
| 30.7 | 2.9 | 9 |
| 31.1 | 2.9 | 6 |
| 32.1 | 2.8 | 4 |
| 32.9 | 2.7 | 3 |
| 35.0 | 2.6 | 4 |
| 35.3 | 2.5 | 4 |
| 36.6 | 2.5 | 3 |
| 40.0 | 2.3 | 3 |

### EXAMPLE 6

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form A.

To a solution of 4-(3,4-dichlorophenoxy)-1,4′-bipiperidine (60 g) in TBF (600 ml) under N₂ at 25° C. was added dropwise a solution of 4-methylbenzenesulfonyl isocyanate (28.43 ml) in THF (250 ml) and the reaction stirred under N₂ at 25° C. for 45 minutes. Aqueous sodium hydroxide (10M, 18.8 ml) was added followed immediately by the addition of water (40 ml). After stirring for 24 hours the precipitate was filtered to give the crude material (73.8 g). To the crude material (10 g) was added water (50 ml) and the mixture heated to 60° C., giving dissolution of the solid. The solution was cooled to 35° C. and stirred for 4 hours, giving precipitation, and then cooled to 20° C. and stirred for 20 hours. The precipitate was filtered to give a solid. This was dried (30° C., 10 mbar) to give the title compound (9.16 g).

NMR data consistent with Example 5.

Contains 8.3% w/w moisture by TGA.

### EXAMPLE 7

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form A.

To a solution of 4-(3,4-dichlorophenoxy)-1,4′-bipiperidine (5 g) in THF (50 ml) under N₂ at 25° C. was added 4-methylbenzenesulfonyl isocyanate (2.32 ml) in THF (20 ml) dropwise and the reaction stirred under N₂ at 25° C. for 3 hours. Water (14 ml) was added and the reaction stirred for 18 hours. The precipitate was now filtered to give N-[[4-(3,4-Dichlorophenoxy)[1,4′-bipiperidin]-1′-yl]carbonyl]-4-methyl-benzenesulfonamide as a solid. To the solid was added water (30 ml) and the mixture heated to 40° C. 10M Aqueous sodium hydroxide, (1.52 ml) was now added and the reaction cooled to 35° C. giving a precipitate. The mixture was stirred for 20 hours and then the precipitate was filtered. The solid was dried (30° C., 10 mbar) to give the title compound (7.31 g).

NMR data consistent with the Example 5

Contains 8.7% w/w moisture by TGA.

### EXAMPLE 8

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form A.

To a solution of 4-(3,4-dichlorophenoxy)-1,4′-bipiperidine (5 g) in THF (50 ml) under N₂ at 25° C. was added 4-methylbenzenesulfonyl isocyanate (2.32 ml) in THF (10 ml) dropwise and the reaction stirred under N₂ at 25° C. for 1 hour. 10M Aqueous sodium hydroxide (1.52 ml) in water (50 ml) was now added and the reaction stirred at 20° C. for 18 hours. THF (60 ml) was removed by distillation (reaction temperature 46-60° C., 500 mbar), and the solution cooled to 35° C., giving precipitation. After stirring at 20° C. for 18 hours the precipitate was filtered. The solid was dried (33° C., 25 mbar) to give the title compound (7.07 g).

NMR data consistent with Example 5.

Contains 8.3% w/w moisture by TGA.

### EXAMPLE 9

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form A.

To a solution of 4-(3,4-dichlorophenoxy)-1,4′-bipiperidine (5 g) in THF (70 ml) under N₂ at 25° C. was added 4-methylbenzenesulfonyl isocyanate (2.33 ml) in one portion. The reaction was stirred under N₂ at 25° C. for 30 minutes. Aqueous sodium hydroxide (10M, 1.52 ml) in water (14 ml) was now added and the reaction stirred for 2 hours. Further 10M aqueous sodium hydroxide (1.52 ml) was added and the reaction stirred at 20° C. for 20 hours. Solvent (50 ml) was removed by distillation (1 bar) and the solution cooled to 20° C. Water (10 ml) was added and the solution stirred for 18 hours giving precipitation. The mixture was filtered and dried (30° C., 9 mbar) to give the title compound (5.35 g).

NMR data consistent with Example 5.

Contains 3.5% w/w moisture by TGA; 3.8% w/w water content by Karl Fischer analysis.

### EXAMPLE 10

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form B.

To a solution of 4-(3,4-dichlorophenoxy)-1,4′-bipiperidine (5 g) in THF (50 ml) under N₂ at 25° C. was added 4-methylbenzenesulfonyl isocyanate (2.33 ml) in THF (20 ml) dropwise and the reaction stirred under N₂ at 25° C. for 15 minutes. 10M Aqueous sodium hydroxide (1.52 ml) was then added and the reaction stirred for 24 hours. The precipitate that formed was filtered to leave a solid that was dried (40° C., 10-30 mbar) to give the title compound (7.04 g).

m.pt. 237° C.

¹H NMR δ (CD₃OD) 1.28-1.42 (2H, m), 1.70-1.82 (4H, m), 1.96-2.04 (2H, m), 2.35 (3H, s), 2.43-2.54 (3H, m), 2.56-2.66 (2H, m), 2.80-2.87 (2H, m), 4.34-4.42 (3H, m), 6.87-6.90 (1H, m), 7.09-7.10 (1H, m), 7.19-7.23 (2H, m), 7.35-7.38 (1H, m), 7.75-7.79 (2H, m). There is a large water peak at 4.87.

¹³C NMR δ (CD₃OD) 21.4, 29.3, 31.6, 43.2, 47.2, 63.8, 74.5, 117.3, 119.1, 124.7, 128.0, 129.7, 132.1, 133.8, 142.0, 144.0, 158.3, 162.8.

Contains 6.52% w/w moisture by TGA; and 6.3% w/w water content by Karl Fischer analysis.

XRPD of Hydrate Form B is presented in FIG. 4. XRPD main reflection peaks are:

| Reflection angle (°2θ) | D-spacing (Å) | Relative intensity (%) |
|---|---|---|
| 4.5 | 19.6 | 82 |
| 4.8 | 18.6 | 28 |
| 7.3 | 12.1 | 16 |
| 8.3 | 10.7 | 99 |
| 11.1 | 7.9 | 4 |
| 13.3 | 6.7 | 16 |
| 14.5 | 6.1 | 56 |
| 14.8 | 6.0 | 51 |
| 15.4 | 5.8 | 41 |
| 16.6 | 5.3 | 85 |
| 17.3 | 5.1 | 9 |
| 18.7 | 4.8 | 26 |
| 19.0 | 4.7 | 14 |
| 19.5 | 4.5 | 29 |
| 20.2 | 4.4 | 100 |
| 21.1 | 4.2 | 37 |
| 21.5 | 4.1 | 39 |
| 21.9 | 4.1 | 70 |
| 22.3 | 4.0 | 27 |
| 23.0 | 3.9 | 9 |
| 23.5 | 3.8 | 46 |
| 24.9 | 3.6 | 24 |
| 25.6 | 3.5 | 3 |
| 26.8 | 3.3 | 7 |
| 27.3 | 3.3 | 16 |
| 27.7 | 3.2 | 9 |
| 28.1 | 3.2 | 11 |
| 28.8 | 3.1 | 11 |
| 29.2 | 3.1 | 15 |
| 30.0 | 3.0 | 10 |
| 31.0 | 2.9 | 5 |
| 31.3 | 2.9 | 5 |
| 31.9 | 2.8 | 9 |
| 32.3 | 2.8 | 10 |
| 32.8 | 2.7 | 7 |
| 33.8 | 2.7 | 6 |
| 34.9 | 2.6 | 7 |
| 35.9 | 2.5 | 5 |
| 37.3 | 2.4 | 7 |
| 38.8 | 2.3 | 9 |
| 39.7 | 2.3 | 2 |

### EXAMPLE 11

This Example illustrates the preparation of N-[[4-(3,4-dichlorophenoxy)[1,4′-bipiperidin]-1′-yl]carbonyl]-4-methyl-benzenesulfonamide Form A.

4-(3,4-Dichlorophenoxy)-1,4′-bipiperidine (4.9 g) was dissolved in dichloromethane (50 ml). 4-Methylbenzenesulfonyl isocyanate (3.8 ml) was added dropwise. The resulting solution was stirred for 1 hour then added to an SCX-2 column (50 g SCX-2 resin; International Sorbent Technology Ltd) and eluted with methanol then methanol-aqueous ammonia (0.88 specific gravity; 9:1). The ammoniacal fractions were evaporated and the residue was stirred with ether for 16 hours. The resultant solid was purified by flash chromatography (dichloromethane:7M ammonia in methanol 6:1) followed by trituration with ether to give a solid. The solid was recrystallized from ethanol and then purified by RPHPLC (Xterra® column; 95:5 to 5:95 aq ammonia:MeCN). Product containing fractions were freeze-dried and then triturated with acetonitrile and finally dried in vacuo at RT to give the title compound (3.1 g;).

m.pt. 233-235° C.

¹H NMR δ (CD₃OD+NaOD) 1.27-1.39 (2H, m), 1.71-1.84 (4H, m), 1.97-2.03 (2H, m), 2.36 (3H, s), 2.44-2.52 (3H, m), 2.58-2.66 (2H, m), 2.79-2.85 (2H, m), 4.35-4.42 (3H, m), 6.88 (1H, dd), 7.08 (1H, d), 7.23 (2H, d), 7.37 (1H, d), 7.76 (2H, d).

No weight loss detected by TGA below melting point.

XRPD of a sample of N-[[4-(3,4-dichlorophenoxy)[1,4′-bipiperidin]-1′-yl]carbonyl]-4-methyl-benzenesulfonamide Form A is presented in FIG. 8. XRPD main reflection peaks are:

| Reflection angle (°2θ) | D-spacing (Å) | Relative intensity (%) |
|---|---|---|
| 7.3 | 12.2 | 6 |
| 8.5 | 10.4 | 22 |
| 10.6 | 8.3 | 7 |
| 13.4 | 6.6 | 8 |
| 14.7 | 6.0 | 18 |
| 15.4 | 5.7 | 17 |
| 15.9 | 5.6 | 100 |
| 16.5 | 5.4 | 13 |
| 16.8 | 5.3 | 8 |
| 17.1 | 5.2 | 15 |
| 18.5 | 4.8 | 13 |
| 18.9 | 4.7 | 5 |
| 19.4 | 4.6 | 8 |
| 19.9 | 4.5 | 27 |
| 20.2 | 4.4 | 30 |
| 20.6 | 4.3 | 4 |
| 20.9 | 4.3 | 3 |
| 21.7 | 4.1 | 29 |
| 22.0 | 4.0 | 7 |
| 22.3 | 4.0 | 2 |
| 23.0 | 3.9 | 3 |
| 23.3 | 3.8 | 8 |
| 23.6 | 3.8 | 19 |
| 24.0 | 3.7 | 4 |
| 24.5 | 3.6 | 3 |
| 25.4 | 3.5 | 4 |
| 25.8 | 3.4 | 19 |
| 26.6 | 3.3 | 18 |
| 27.0 | 3.3 | 4 |
| 27.6 | 3.2 | 5 |
| 28.7 | 3.1 | 8 |
| 29.5 | 3.0 | 6 |
| 31.5 | 2.8 | 6 |
| 32.4 | 2.8 | 2 |
| 33.1 | 2.7 | 3 |
| 33.8 | 2.7 | 5 |
| 39.5 | 2.3 | 2 |

### EXAMPLE 12

This Example illustrates the preparation of N-[[4-(3,4-dichlorophenoxy)[1,4′-bipiperidin]-1′-yl]carbonyl]-4-methyl-benzenesulfonamide Form A.

To N-[[4-(3,4-dichlorophenoxy)[1,4′-bipiperidin]-1′-yl]carbonyl]A-methyl-benzenesulfonamide Form B (see Example 13; 4.89 g) was added acetonitrile (50 ml) and the mixture heated to 50° C. Further acetonitrile (50 ml) was added and the resultant slurry was stirred overnight at 50° C. The heater was then turned off and the flask allowed to cool to room temperature in the oil bath. The slurry was filtered and the resultant solid dried in vacuo overnight at 40° C. to give the title compound (4.45 g)

XRPD data consistent with Example 11.

### EXAMPLE 13

This Example illustrates the preparation of N-[[4-(3,4-dichlorophenoxy)[1,4′-bipiperidin]-1′-yl]carbonyl]-4-methyl-benzenesulfonamide Form B.

4-(3,4-Dichlorophenoxy)-1,4′-bipiperidine (5.0 g) was dissolved in dichloromethane (40 ml). 4-Methylbenzenesulfonyl isocyanate (3.3 ml) was added dropwise. The mixture was stirred for 30 minutes and then water was added. The layers were separated and the organic phase was allowed to stand whilst product crystallized. The solid was collected, washed with dichloromethane and dried in vacuo at 40° C. to give the title compound (6.2 g;).

m.pt. 207-212° C.

¹H NMR δ(CD₃OD+NaOD): 1.27-1.40 (2H, m), 1.70-1.84 (4H, m), 1.96-2.05 (2H, m), 2.36 (3H, s), 2.44-2.52 (3H, m), 2.56-2.67 (2H, m), 2.79-2.86 (2H, m), 4.34-4.43 (3H, m), 6.88 (1H, dd), 7.09 (1H, d), 7.22 (2H, d), 7.37 (1H, d), 7.74-7.77 (2H, m) Contains 0.11% w/w moisture by TGA.

XRPD of a sample of N-[[4-(3,4-dichlorophenoxy)[1,4′-bipiperidin]-1′-yl]carbonyl]-4-methyl-benzenesulfonamide Form B is presented in FIG. 9. XRPD main reflection peaks are:

| Reflection angle (°2θ) | D-spacing (Å) | Relative intensity (%) |
|---|---|---|
| 5.4 | 16.5 | 38 |
| 6.1 | 14.5 | 12 |
| 9.9 | 9.0 | 21 |
| 10.4 | 8.5 | 23 |
| 10.5 | 8.4 | 24 |
| 11.0 | 8.0 | 84 |
| 11.6 | 7.6 | 75 |
| 12.9 | 6.9 | 14 |
| 13.3 | 6.7 | 58 |
| 13.9 | 6.4 | 40 |
| 14.9 | 6.0 | 75 |
| 16.4 | 5.4 | 9 |
| 17.3 | 5.1 | 26 |
| 18.0 | 4.9 | 38 |
| 19.0 | 4.7 | 55 |
| 19.7 | 4.5 | 26 |
| 20.4 | 4.4 | 100 |
| 21.5 | 4.1 | 32 |
| 22.2 | 4.0 | 85 |
| 23.0 | 3.9 | 50 |
| 23.3 | 3.8 | 43 |
| 23.8 | 3.7 | 20 |
| 25.2 | 3.5 | 11 |
| 26.7 | 3.3 | 5 |
| 27.7 | 3.2 | 18 |
| 28.2 | 3.2 | 20 |
| 29.0 | 3.1 | 6 |
| 30.2 | 3.0 | 16 |
| 36.5 | 2.5 | 9 |

### EXAMPLE 14

This Example illustrates the preparation of N-[[4-(3,4-dichlorophenoxy)[1,4′-bipiperidin]-1′-yl]carbonyl]-4-methyl-benzenesulfonamide Form B.

To a solution of 4-(3,4-dichlorophenoxy)-1,4′-bipiperidine (7.93 g) in DCM (50 ml) under N₂ at room temperature was added dropwise a solution of 4-methylbenzenesulfonyl isocyanate (3.68 ml) in DCM (25 ml). The resultant solution was stirred at room temperature for three hours during which time precipitation occurred. The resultant solid was filtered and washed with DCM (80 ml). The damp solid was dried in vacuo at 35° C. overnight to give the title compound (9.63 g).

XRPD data is consistent with the Example 13.

### EXAMPLE 15

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form C.

To the sodium salt of N-[[4-(3,4-dichlorophenoxy)[1,4′-bipiperidin]-1′-yl]carbonyl]-4-methyl-benzenesulfonamide (2.93 g) was added water (6 ml) and acetone (24 ml) and the resultant slurry heated to reflux to obtain a solution. The solution was allowed to cool to room temperature and then cooled further with ice/water. The resultant slurry was filtered and then dried in vacuo overnight at 35° C. to give the title compound (1.96 g).

¹H NMR δ (CD₃OD): 1.28-1.42 (2H, m), 1.70-1.82 (4H, m), 1.96-2.04 (2H, m), 2.35 (3H, s), 2.43-2.54 (3H, m), 2.56-2.66 (2H, m), 2.80-2.87 (2H, m), 4.34-4.42 (3H, m), 6.87-6.90 (1H, m), 7.09-7.10 (1H, m), 7.19-7.23 (2H, m), 7.35-7.38 (1H, m), 7.75-7.79 (2H, m); There is a large water peak at 4.87.

XRPD of a sample of Hydrate Form C is presented in FIG. 5. XRPD main reflection peaks are:

| Reflection angle (°2θ) | D-spacing (Å) | Relative intensity (%) |
|---|---|---|
| 4.2 | 21.2 | 100 |
| 7.5 | 11.7 | 4 |
| 8.0 | 11.0 | 11 |
| 10.1 | 8.8 | 3 |
| 11.4 | 7.8 | 7 |
| 11.5 | 7.7 | 7 |
| 12.5 | 7.1 | 23 |
| 13.1 | 6.8 | 5 |
| 15.1 | 5.9 | 6 |
| 15.6 | 5.7 | 13 |
| 15.8 | 5.6 | 14 |
| 16.6 | 5.4 | 3 |
| 16.9 | 5.2 | 5 |
| 17.2 | 5.1 | 6 |
| 17.8 | 5.0 | 9 |
| 18.9 | 4.7 | 14 |
| 19.9 | 4.5 | 7 |
| 20.5 | 4.3 | 15 |
| 21.1 | 4.2 | 11 |
| 21.6 | 4.1 | 4 |
| 22.7 | 3.9 | 7 |
| 23.8 | 3.7 | 4 |
| 24.6 | 3.6 | 16 |
| 25.1 | 3.5 | 5 |
| 26.1 | 3.4 | 5 |
| 27.8 | 3.2 | 6 |
| 29.2 | 3.1 | 9 |
| 29.5 | 3.0 | 8 |
| 30.2 | 3.0 | 3 |
| 30.5 | 2.9 | 3 |
| 31.7 | 2.8 | 7 |
| 32.0 | 2.8 | 5 |
| 32.7 | 2.7 | 3 |
| 33.5 | 2.7 | 2 |

### EXAMPLE 16

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form A.

A solution of 4-(3,4-dichlorophenoxy)-1,4′-bipiperidine (5 g) in chlorobenzene (50 ml) was distilled under vacuum to remove solvent (15 ml). To this solution, under N₂ at 25° C., was added 4-methylbenzenesulfonyl isocyanate (2.32 ml) in chlorobenzene (5 ml) in one portion. Chlorobenzene (2.5 ml) was used to wash in the reagent. After 30 minutes 10M aqueous sodium hydroxide (1.67 ml) in water (40 ml) was added. The reaction was heated to 70° C. and the layers allowed to separate. After separation, the aqueous phase was washed with toluene (25 ml) and then collected. To induce crystallization water (15 ml), IMS 74 OP (27 ml) and toluene (3.66 ml) were added to the aqueous phase and the mixture cooled to 5° C. After stirring at 5° C. for 18 hours the precipitate was filtered. The solid was immediately dried (33° C., 25 mbar) to give the title compound (8.17 g).

XRPD consistent with Example 5.

Contains 9.06% w/w moisture by TGA.

### EXAMPLE 17

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form A.

A solution of 4-(3,4-dichlorophenoxy)-1,4′-bipiperidine (5 g) in toluene (50 ml) was distilled under vacuum to remove solvent (27 ml). The solution was cooled to 40° C. under N₂ and tetrahydrofuran (25 ml) added. To this solution was added 4-methylbenzenesulfonyl isocyanate (2.32 ml) in tetrahydrofuran (5 ml) in one portion. After 30 minutes 10M aqueous sodium hydroxide (1.67 ml) in water (40 ml) was added. Toluene (15 ml) was added and then solvent (20 ml) distilled under vacuum. The reaction mixture was heated to 60° C. and the layers allowed to separate. After separation, to the aqueous was added water (15 ml), IMS 74 OP (25 ml) and the reaction cooled to 5° C. After stirring at 5° C. for 20 hours the precipitate was filtered and washed with water (10 ml)/IMS 74 OP (5 ml) mixture. The solid was immediately dried (33° C., 25 mbar) to give the title compound (8.15 g).

XRPD consistent with Example 5.

### EXAMPLE 18

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form D.

To the sodium salt of N-[[4-(3,4-dichlorophenoxy)[1,4′-bipiperidin]-1′-yl]carbonyl]-4-methyl-benzenesulfonamide (8 g) was added water (24 ml) and 2-propanol (24 ml) and the resultant slurry heated to 70° C. to obtain a solution. The solution was allowed to cool to 5° C. over 2 hours 10 minutes (0.5° C./min). The resultant slurry was stirred for four days before approximately half the material was drained from the vessel. The slurry was filtered and washed with water (30 ml). A portion of the damp cake was packed into a vial and stored for 5 days to give a damp cake containing the title compound.

XRPD of a sample of Hydrate Form D is presented in FIG. 6. XRPD main reflection peaks are:

| Reflection angle (° 2θ) | D-spacing (Å) | Relative intensity (%) |
|---|---|---|
| 8.8 | 10.0 | 12 |
| 10.5 | 8.4 | 9 |
| 11.7 | 7.6 | 29 |
| 11.8 | 7.5 | 33 |
| 12.8 | 6.9 | 10 |
| 12.9 | 6.8 | 10 |
| 15.6 | 5.7 | 15 |
| 16.5 | 5.4 | 11 |
| 17.0 | 5.2 | 14 |
| 117.1 | 5.2 | 17 |
| 18.9 | 4.7 | 14 |
| 18.9 | 4.7 | 13 |
| 19.1 | 4.6 | 10 |
| 20.8 | 4.3 | 38 |
| 21.1 | 4.2 | 24 |
| 22.1 | 4.0 | 17 |
| 22.1 | 4.0 | 17 |
| 22.5 | 3.9 | 14 |
| 22.7 | 3.9 | 20 |
| 22.8 | 3.9 | 21 |
| 22.9 | 3.9 | 21 |
| 23.3 | 3.8 | 100 |
| 25.6 | 3.5 | 19 |
| 25.6 | 3.5 | 22 |
| 26.1 | 3.4 | 20 |
| 26.1 | 3.4 | 21 |
| 26.9 | 3.3 | 29 |
| 28.1 | 3.2 | 25 |
| 28.5 | 3.1 | 12 |
| 29.3 | 3.0 | 14 |
| 29.5 | 3.0 | 12 |
| 30.0 | 3.0 | 12 |
| 30.1 | 3.0 | 13 |
| 30.1 | 3.0 | 16 |
| 30.3 | 2.9 | 18 |
| 30.5 | 2.9 | 21 |
| 30.6 | 2.9 | 23 |
| 30.8 | 2.9 | 18 |
| 32.5 | 2.8 | 20 |
| 33.1 | 2.7 | 20 |

### EXAMPLE 19

This Example illustrates the preparation of a form of the sodium salt of N-[[4-(3,4-dichlorophenoxy)[1,4′-bipiperidin]-1′-yl]carbonyl]-4-methyl-benzenesulfonamide herein referred to as Solvated Form E.

To the sodium salt of N-[[4-(3,4-dichlorophenoxy)[1,4′-bipiperidin]-1′-yl]carbonyl]-4-methyl-benzenesulfonamide (164 g) was added water (984 ml), IMS 74 OP (492 ml) and toluene (73.8 ml) and the resulting mixture heated to 70° C. to give a solution. The mixture was cooled to 5° C. and the material stirred overnight. Approximately half of the resultant slurry was filtered and washed with a 5° C. 2:1 mixture of water/IMS 74 OP (150 ml) to give 265 g of a damp cake containing the title compound.

Analysis of the cake shows it contains 71.8% w/w of solvents by TGA.

XRPD of a sample of Solvated Form E is presented in FIG. 7. XRPD main reflection peaks are:

| Reflection angle (° 2θ) | D-spacing (Å) | Relative intensity (%) |
|---|---|---|
| 3.64 | 24.25 | 45 |
| 7.10 | 12.44 | 18 |
| 8.30 | 10.64 | 21 |
| 9.34 | 9.46 | 28 |
| 9.78 | 9.04 | 19 |
| 12.16 | 7.27 | 19 |
| 14.06 | 6.29 | 28 |
| 14.26 | 6.21 | 24 |
| 15.86 | 5.58 | 34 |
| 17.68 | 5.01 | 35 |
| 18.56 | 4.78 | 100 |
| 19.34 | 4.59 | 49 |
| 19.52 | 4.54 | 48 |
| 20.40 | 4.35 | 41 |
| 20.48 | 4.33 | 41 |
| 20.60 | 4.31 | 40 |
| 21.16 | 4.20 | 33 |
| 21.60 | 4.11 | 52 |
| 21.72 | 4.09 | 63 |
| 22.44 | 3.96 | 36 |
| 23.06 | 3.85 | 59 |
| 24.06 | 3.70 | 59 |
| 24.16 | 3.68 | 51 |
| 24.96 | 3.56 | 66 |
| 25.06 | 3.55 | 55 |
| 25.18 | 3.53 | 47 |
| 25.46 | 3.50 | 46 |
| 25.76 | 3.46 | 67 |
| 25.82 | 3.45 | 55 |
| 26.28 | 3.39 | 72 |
| 26.68 | 3.34 | 45 |
| 27.10 | 3.29 | 43 |
| 27.18 | 3.28 | 45 |
| 27.34 | 3.26 | 46 |
| 27.92 | 3.19 | 62 |
| 28.58 | 3.12 | 44 |
| 28.68 | 3.11 | 51 |
| 28.92 | 3.08 | 51 |
| 29.06 | 3.07 | 45 |
| 29.22 | 3.05 | 50 |
| 29.36 | 3.04 | 49 |
| 29.42 | 3.03 | 52 |
| 29.78 | 3.00 | 52 |
| 29.88 | 2.99 | 56 |
| 30.16 | 2.96 | 46 |
| 30.28 | 2.95 | 53 |
| 32.58 | 2.75 | 48 |

### EXAMPLE 20

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form A.

After isolation a damp cake of sodium salt of N-[[4-(3,4-dichlorophenoxy)[1,4′-bipiperidin]-1′-yl]carbonyl]-4-methyl-benzenesulfonamide Hydrate Form D (see Example 18) was transferred immediately to a vacuum oven and dried in vacuo at 35° C. to give the title compound.

XRPD consistent with Example 5.

### EXAMPLE 21

This Example illustrates the preparation of the sodium salt of N-[[4-(3,4-dichlorophenoxy)[1,4′-bipiperidin]-1′-yl]carbonyl]-4-methyl-benzenesulfonamide as Hydrate Form A.

Solvent from a damp cake of sodium salt of N-[[4-(3,4-dichlorophenoxy)[1,4′-bipiperidin]-1′-yl]carbonyl]-4-methyl-benzenesulfonamide Solvated Form E (see Example 19) was removed by filtration. The resultant material was immediately transferred to a Petri dish to give a thin cake and allowed to dry within a fume cupboard to give the title compound.

XRPD consistent with Example 5.

### EXAMPLE 22

This Example illustrates the preparation of the sodium salt of N-[[4-3,4-dichlorophenoxy)[1,4′-bipiperidin]-1′-yl]carbonyl]-4-methyl-benzenesulfonamide as Hydrate Form C.

A damp cake of sodium salt of N-[[4-(3,4-dichlorophenoxy)[1,4′-bipiperidin]-1′-yl]carbonyl]-4-methyl-benzenesulfonamide Solvated Form E (see Example 19) was stored in a sealed vacuum oven over the weekend. The material was then dried in the oven at 35° C. and 100 mbar to give the title compound.

XRPD consistent with Example 15.

### EXAMPLE 23

### Pharmacological Analysis: Calcium Flux [Ca²⁺]ᵢ assay

Human Eosinophils

Human eosinophils were isolated from EDTA anticoagulated peripheral blood as previously described (Hansel et al., J. Immunol. Methods, 1991, 145, 105-110). The cells were resuspended (5×10⁶ ml⁻¹) and loaded with 5 μM FLUO-3/AM+Pluronic F127 2.2 μl/ml (Molecular Probes) in low potassium solution (LKS; NaCl 118 mM, MgSO₄ 0.8 mM, glucose 5.5 mM, Na₂CO₃ 8.5 mM, KCl 5 mM, BEPES 20 mM, CaCl₂ 1.8 mM, BSA 0.1%, pH 7.4) for one hour at room temperature. After loading, cells were centrifuged at 200 g for 5 min and resuspended in LKS at 2.5×10⁶ ml⁻¹. The cells were then transferred to 96 well FLIPr plates (Poly-D-Lysine plates from Becton Dickinson pre-incubated with 5 μM fibronectin for two hours) at 25 μl/well. The plate was centrifuged at 200 g for 5 min and the cells were washed twice with LKS (200 μl; room temperature).

A compound of the Examples was pre-dissolved in DMSO and added to a final concentration of 0.1% (v/v) DMSO. Assays were initiated by the addition of an A₅₀ concentration of eotaxin and the transient increase in fluo-3 fluorescence (l_{Ex}=490 nm and l_{Em}=520 nm) monitored using a FLIPR (Fluorometric Imaging Plate Reader, Molecular Devices, Sunnyvale, U.S.A.).

### Human Eosinophil Chemotaxis

Human eosinophils were isolated from EDTA anticoagulated peripheral blood as previously described (Hansel et al., J. Immunol. Methods, 1991, 45, 105-110). The cells were resuspended at 10×10⁶ ml⁻¹ in RPMI containing 200 IU/ml penicillin, 200 μg/ml streptomycin sulfate and supplemented with 10% HIFCS, at room temperature.

Eosinophils (700 μl) were pre-incubated for 15 mins at 37° C. with 7 μl of either vehicle or compound (100× required final concentration in 10% DMSO). The chemotaxis plate (ChemoTx, 3 μm pore, Neuroprobe) was loaded by adding 28 μl of a concentration of eotaxin (0.1 to 100 nM) containing a concentration of a compound according to the Examples or solvent to the lower wells of the chemotaxis plate. The filter was then placed over the wells and 25 μl of eosinophil suspension were added to the top of the filter. The plate was incubated for 1 hr at 37° C. in a humidified incubator with a 95% air/5% CO₂ atmosphere to allow chemotaxis.

The medium, containing cells that had not migrated, was carefully aspirated from above the filter and discarded. The filter was washed once with phosphate buffered saline (PBS) containing 5 mM EDTA to remove any adherent cells. Cells that had migrated through the filter were pelleted by centrifugation (300×g for 5 mins at room temperature) and the filter removed and the supernatant transferred to each well of a 96-well plate (Costar). The pelleted cells were lysed by the addition of 28 μl of PBS containing 0.5% Triton ×100 followed by two cycles of freeze/thawing. The cell lysate was then added to the supernatant. The number of eosinophils migrating was quantified according to the method of Strath et al., J. Immunol. Methods, 1985, 83, 209 by measuring eosinophil peroxidase activity in the supernatant.

Compounds of the Examples were found to be antagonists of the eotaxin mediated human eosinophil chemotaxis.

### EXAMPLE 24

Histamine H1 receptor binding activity of compounds of the invention was assessed by competition displacement of 1 nM [3H]-pyrilamine (Amersham, Bucks, Product code TRK 608, specific activity 30 Ci/mmol) to 2 μg membranes prepared from recombinant CHO-K1 cells expressing the human H1 receptor (Euroscreen SA, Brussels, Belgium, product code ES-390-M) in assay buffer (50 mM Tris pH 7.4 containing 2 mM MgCl₂, 250 mM sucrose and 100 mM NaCl) for 1 hour at room temperature.

| Example | H1 pKi/[1328_S] |
|---|---|
| 1 | 7.7 |

1. An anhydrous form of sodium salt of N-[[4-(3,4-dichlorophenoxy)[1,4′-bipiperidin]-1′-yl]carbonyl]-4-methyl-benzenesulfonamide (Anhydrous Form B) having an X-ray powder diffraction pattern containing specific peaks at: 3.8 (±0.1°), 7.5 (±0.1°), 11.2 (±0.1°), 13.0 (±0.1°), 13.8 (±0.1°), 15.0 (±0.1°), 15.7 (±0.1°), 18.8 (±0.1°), 20.2 (±0.1°), 21.7 (±0.1°), 22.6 (±0.1°) and 30.2 (±0.1°) 2θ.

2. An anhydrous form of sodium salt of N-[[4-(3,4-dichlorophenoxy)[1,4′-bipiperidin]-1′-yl]carbonyl]-4-methyl-benzenesulfonamide (Anhydrous Form C) having an X-ray powder diffraction pattern containing specific peaks at: 4.3 (±0.1°), 8.5 (±0.1°), 14.6 (±0.1°), 15.3 (±0.1°), 16.1 (±0.1°), 17.4 (±0.1°), 18.7 (±0.1°), 20.5 (±0.1°), 22.1 (±0.1°), 22.6 (±0.1°), 23.1 (±0.1°) and 29.6 (±0.1°) 2θ.

3. A hydrated form of sodium salt of N-[[4-(3,4-dichlorophenoxy)[1,4′-bipiperidin]-1′-yl]carbonyl]-4-methyl-benzenesulfonamide (Hydrate Form A) having an X-ray powder diffraction pattern containing specific peaks at: 4.2 (±0.1°), 8.2 (±0.1°), 8.5 (±0.1°), 9.1 (±0.1°), 11.5 (±0.1°), 12.7 (±0.1°), 14.8 (±0.1°), 15.4 (±0.1°), 16.6 (±0.1°), 17.4 (±0.1°), 17.7 (±0.1°), 18.2 (±0.1°), 20.4 (±0.1°), 23.2 (±0.1°), 29.1 (±0.1°) and 29.8 (±0.1°) 2θ.

4. A compound as claimed in claim 3 wherein the water of crystallization is 3-10% w/w.

5. A hydrated form of sodium salt of N-[[4-(3,4-dichlorophenoxy)[1,4′-bipiperidin]-1′-yl]carbonyl]-4-methyl-benzenesulfonamide (Hydrate Form B) having an X-ray powder diffraction pattern containing specific peaks at: 4.5 (±0.1°), 7.3 (±0.1°), 8.3 (±0.1°), 13.3 (±0.1°), 14.5 (±0.1°), 14.8 (±0.1°), 15.4 (±0.1°), 16.6 (±0.1°), 18.7 (±0.1°), 20.2 (±0.1°), 21.1 (±0.1°), 21.5 (±0.1°), 21.9 (±0.1°), 22.3 (±0.1°), 23.5 (±0.1°) and 24.9 (±0.1°) 2θ.

6. A compound as claimed in claim 5 wherein the water of crystallization is 5-7% w/w.

7. A hydrated form of the sodium salt of N-[[4-(3,4-dichlorophenoxy)[1,4′-bipiperidin]-1′-yl]carbonyl]-4-methyl-benzenesulfonamide (Hydrate Form C) having an X-ray powder diffraction pattern containing specific peaks at: 4.2 (±0.1°), 7.5 (±0.1°), 8.0 (±0.1°), 11.4 (±0.1°), 12.5 (±0.1°), 15.1 (±0.1°), 15.8 (±0.1°), 17.7 (±0.1°), 18.9 (±0.1°), 20.5 (±0.1°), 21.1 (±0.1°), 22.7 (±0.1°), 24.6 (±0.1°), 26.1 (±0.1°), 27.8 (±0.1°) and 29.2 (±0.1°) 2θ.

8. A compound as claimed in claim 7 wherein the water of crystallization is 3-10% w/w.

9. A hydrated form of the sodium salt of N-[[4-(3,4-dichlorophenoxy)[1,4′-bipiperidin]-1′-yl]carbonyl]-4-methyl-benzenesulfonamide (Hydrate Form D) having an X-ray powder diffraction pattern containing specific peaks at: 8.8 (±0.1°), 10.5 (±0.1°), 11.8 (±0.1°), 12.9 (±0.1°), 15.6 (±0.1°), 17.1 (±0.1°), 18.9 (±0.1°), 20.8 (±0.1°), 23.3 (±0.1°), 25.6 (±0.1°), 26.1 (±0.1°), 26.9 (±0.1°), 28.1 (±0.1°), 30.6 (±0.1°), 32.5 (±0.1°) and 33.1 (±0.1°) 2θ.

10. A solvated form of the sodium salt of N-[[4-(3,4-dichlorophenoxy)[1,4′-bipiperidin]-1′-yl]carbonyl]-4-methyl-benzenesulfonamide (Solvated Form E) having an X-ray powder diffraction pattern containing specific peaks at: 3.6 (±0.1°), 7.1 (±0.1°), 8.3 (±0.1°), 9.3 (±0.1°), 9.8 (±0.1°), 14.1 (±0.1°), 15.9 (±0.1°), 17.7 (±0.1°), 18.6 (±0.1°), 19.3 (±0.1°), 21.7 (±0.1°), 23.1 (±0.1°), 24.1 (±0.10), 25.0 (±0.1°), 25.8 (±0.1°) and 26.3 (±0.1°) 2θ.

11. A crystalline form of N-[[4-(3,4-dichlorophenoxy)[1,4′-bipiperidin]-1′-yl]carbonyl]-4-methyl-benzenesulfonamide (Form A) having an X-ray powder diffraction pattern containing specific peaks at: 7.3 (±0.1°), 8.5 (±0.1°), 10.6 (±0.1°), 13.4 (±0.1°), 14.7 (±0.1°), 15.4 (±0.1°), 15.9 (±0.1°), 19.9 (±0.1°), 20.2 (±0.1°), 21.7 (±0.1°), 25.8 (±0.1°) and 26.6 (±0.1°) 2θ.

12. A crystalline form of N-[[4-(3,4-dichlorophenoxy)[1,4′-bipiperidin]-1′-yl]carbonyl]-4-methyl-benzenesulfonamide (Form B) having an X-ray powder diffraction pattern containing specific peaks at: 9.9 (±0.1°), 10.5 (±0.1°), 11.0 (±0.1°), 11.6 (±0.1°), 13.3 (±0.1°), 13.9 (±0.1°), 14.9 (±0.1°), 18.0 (±0.1°), 19.0 (±0.1°), 20.4 (±0.1°), 22.2 (±0.1°) and 23.0 (±0.1°) 2θ.

13-16. (canceled)

17. A process for preparing Anhydrous Form B comprising:
a. drying a water-wet or hydrated form of a sample of the sodium salt of N-[[4-(3,4-dichlorophenoxy)[1,4′-bipiperidin]-1′-yl]carbonyl]-4-methyl-benzenesulfonamide in the presence of phosphorus pentoxide under reduced pressure; or,
b. heating a sample of Hydrate Form A from ambient temperature to 100° C.

18. A process for preparing Anhydrous Form C comprising heating a sample of Hydrate Form B from ambient temperature to 100° C.

19. A process for preparing Hydrate Form A comprising reacting 4-(3,4-dichlorophenoxy)-1,4′-bipiperidine with 4-methylbenzenesulfonyl isocyanate in a suitable solvent at ambient temperature to form N-[[4-(3,4-dichlorophenoxy)[1,4′-bipiperidin]-1′-yl]carbonyl]-4-methyl-benzenesulfonamide in the suitable solvent; adding to that concentrated aqueous sodium hydroxide solution followed by water; and:
a. stirring the resulting mixture to allow the sodium salt of N-[[4-(3,4-dichlorophenoxy)[1,4′-bipiperidin]-1′-yl]carbonyl]-4-methyl-benzenesulfonamide, possibly contaminated with suitable solvent, to precipitate out with Hydrate Form A remaining after filtration and drying, or,
b. distilling the suitable solvent and allowing Hydrate Form A to precipitate from the aqueous.

20. A process for preparing Hydrate Form A comprising adding concentrated aqueous sodium hydroxide solution to a mixture of N-[[4-(3,4-dichlorophenoxy)[1,4′-bipiperidin]-1′-yl]carbonyl]-4-methyl-benzenesulfonamide in water at a temperature in the range 30-60° C. and allowing the mixture to cool with the sodium salt of N-[[4-(3,4-dichlorophenoxy)[1,4′-bipiperidin]-1′-yl]carbonyl]-4-methyl-benzenesulfonamide precipitating and Hydrate Form A remaining after filtering and drying.

21. A process for preparing Hydrate Form A as claimed in claim 20 comprising:
a. mixing N-[[4-(3,4-dichlorophenoxy)[1,4′-bipiperidin]-1′-yl]carbonyl]-4-methyl-benzenesulfonamide with water and heating the mixture to a temperature in the range 30-60° C.; and,
b. adding concentrated aqueous sodium hydroxide solution and allowing the mixture to cool with the sodium salt of N-[[4-(3,4-dichlorophenoxy)[1,4′-bipiperidin]-1′-yl]carbonyl]-4-methyl-benzenesulfonamide precipitating and Hydrate Form A remaining after filtering and drying.

22. A process for preparing Hydrate Form A comprising adding concentrated aqueous sodium hydroxide solution to N-[[4-(3,4-dichlorophenoxy)[1,4′-bipiperidin]-1′-yl]carbonyl]-4-methyl-benzenesulfonamide in a suitable organic solvent; heating the mixture and separating the aqueous layer; adding IMS and, optionally, toluene to the aqueous phase and cooling the resulting mixture; and, filtering off and drying the solid that forms.

23. A process for preparing Hydrate Form A comprising heating a mixture of N-[[4-(3,4-dichlorophenoxy)[1,4′-bipiperidin]-1′-yl]carbonyl]-4-methyl-benzenesulfonamide (Form B) and aqueous sodium hydroxide; cooling the mixture and extracting the cooled mixture with-dichloromethane; combining the extracts; optionally reducing the volume of the combined organic extracts; cooling the dichloromethane mixture so that the sodium salt of N-[[4-(3,4-dichlorophenoxy)[1,4′-bipiperidin]-1′-yl]carbonyl]-4-methyl-benzenesulfonamide precipitates; and, filtering off and drying the solid that forms.

24. A process for preparing Hydrate Form A comprising drying a sample of Hydrate Form D under reduced pressure at a temperature in the range 10-100° C.

25. A process for preparing Hydrate Form A comprising drying a sample of Solvated Form E at atmospheric pressure at a temperature in the range 0-30° C.

26. A process for preparing Hydrate Form B comprising mixing a solution of 4-(3,4-dichlorophenoxy)-1,4′-bipiperidine in tetrahydrofuran with a solution of 4-methylbenzenesulfonyl isocyanate in tetrahydrofuran at a temperature in the range 15-35° C.; adding aqueous sodium hydroxide solution and collecting the solid that precipitates.

27. A process for preparing Hydrate Form- C comprising cooling a solution of the sodium salt of N-[[4-(3,4-dichlorophenoxy)[1,4′-bipiperidin]-1′-yl]carbonyl]-4-methyl-benzenesulfonamide in a mixture of water and acetone from reflux to around 0° C. and collecting the solid product that forms.

28. A process for preparing Hydrate Form C comprising drying a sample of Solvated Form E reduced pressure at a temperature in the range 10-100° C.

29. A process for preparing Hydrate Form D comprising cooling a solution of the sodium salt of N-[[4-(3,4-dichlorophenoxy)[1,4′-bipiperidin]-1′-yl]carbonyl]-4-methyl-benzenesulfonamide in a mixture of water and 2-propanol from 50-80° C. to 0-10° C. and filtering off the residue.

30. A process for preparing Solvated Form E comprising cooling a solution of the sodium salt of N-[[4-(3,4-dichlorophenoxy)[1,4′-bipiperidin]-1′-yl]carbonyl]-4-methyl-benzenesulfonamide in a mixture of water, IMS and toluene from 50-80° C. to 0-10° C. and filtering off the residue.

31. A process for preparing N-[[4-(3,4-Dichlorophenoxy)[1,4′-bipiperidin]-1′-yl]carbonyl]-4-methyl-benzenesulfonamide (Form A) comprising:
a. purifying N-[[4-(3,4-dichlorophenoxy)-[1,4′-bipiperidin]-1′-yl]carbonyl]-4-methyl-benzenesulfonamide using reverse phase chromatography eluting with a mixture of aqueous ammonia and acetonitrile; and,
b. freeze drying the fractions containing N-[[4-(3,4-dichlorophenoxy)-[1,4′-bipiperidin]-1′-yl]carbonyl]-4-methyl-benzenesulfonamide and triturating the residue with acetonitrile and then drying the residue under reduced pressure at ambient temperature.

32. A process for preparing N-[[4-(3,4-dichlorophenoxy)[1,4′-bipiperidin]-1′-yl]carbonyl]-4-methyl-benzenesulfonamide (Form A) comprising:
a. heating a mixture of N-[[4-(3,4-dichlorophenoxy)[1,4′-bipiperidin]-1′-yl]carbonyl]-4-methyl-benzenesulfonamide Form B and acetonitrile to 40-60° C.; and,
b. drying the solid from the slurry so formed under reduced pressure.
